(19) United States
(12) Patent Application Publication
(10) Pub. No.: US 2026/0224349 A1
(43) Pub. Date: Aug. 6, 2026

(54) CORNEAL IMPLANT

(71) Applicant: Recornea S.r.l., Martignacco (UD) (IT)

(72) Inventors: Edoardo GROSSO, Moncalieri (TO) (IT); Emiliano LEPORE, Trieste (TS) (IT)

(21) Appl. No.: 19/155,609

(22) PCT Filed: Nov. 30, 2023
(86) PCT No.: PCT/IB2023/062065

§ 371 Date: Aug. 12, 2025

(30) Foreign Application Priority Data
Feb. 13, 2023 (IT) 102023000002418

(51) Int. Cl. A61F 2/14

(52) U.S. Cl. CPC A61F 2/142; A61F 2240/001

(57) ABSTRACT

A precursor (100) for corneal implant (1) comprising a structural body (2), the structural body (2) comprising an outer peripheral ring (10), an inner peripheral ring (11), and a connection structure (20) pre-arranged to connect the outer peripheral ring (10) and the inner peripheral ring (11), wherein said outer peripheral ring (10), said inner peripheral ring (11) and said connection structure (20) lie in one and the same plane, and wherein said outer peripheral ring (10) and said inner peripheral ring (11) lie at a first distance apart. The connection structure is designed to undergo a deformation to obtain a predefined dome-like conformation of said structural body (2), wherein said outer peripheral ring (10) lies in a first plane and said inner peripheral ring (11) lies in a second plane distinct from and parallel to said first plane and at a second distance apart from said outer peripheral ring (10) greater than said first distance. The connection structure (20) comprises at least one connection element (23) for connecting the outer peripheral ring to the inner peripheral ring that extends along a profile (22) that deviates from a connection path of minimum length, equal to said first distance, so as to have a length in excess of said first distance that is such as to cover said second distance between said outer peripheral ring and said inner peripheral ring in said dome-like conformation of said structural body.

### FIELD OF THE INVENTION

The present disclosure regards a corneal implant designed to correct irregularities of the curvature of the cornea of a subject.

### BACKGROUND OF THE INVENTION

A corneal implant is an implantable device designed to impose a substantially regular curvature upon the cornea so as to restore an optimal optical situation designed to reduce or eliminate optical aberrations that may jeopardize, for example, visual acuity or sensitivity to contrast.

Treatments that are known and used for treating corneal aberrations induced by curvature irregularities may comprise, for example, i) use of contact lenses, ii) use of ICRS (IntraCorneal Ring Segments), i.e., segments of circular rings made of polymeric material of variable diameter and thickness, iii) ablation treatments with excimer laser, and iv) treatments with techniques of incisional refractive surgery. Such techniques may, however, present some disadvantages linked, for example, to the onset of infections and/or to the intolerance in the case of advanced keratoconus when contact lenses are used; ICRS may have a limited capacity to impose a regular curvature upon the cornea; ablation treatments may lead to an only superficial remodeling of the cornea; incisional refractive surgery may give rise to high risks of inducing secondary corneal ectasia.

Starting from the 1980s different types of corneal implants have been developed for correcting the curvature of the cornea that could overcome the disadvantages of known techniques. Corneal implants capable of imposing a predefined curvature upon the cornea and hence treating pathological deformations are, for example, described in the document EP 3 801 386 A1.

### SUMMARY OF THE INVENTION

Meliorative and effective solutions of corneal implants that will enable an even more satisfactory treatment of irregularities of corneal curvature are at present a subject of interest.

According to the invention, the above object is achieved thanks to the solution specifically recalled in the annexed claims, which form an integral part of the present disclosure.

An embodiment of the present disclosure regards a corneal implant, a precursor for a corneal implant, and the implant deriving therefrom following upon deformation of the precursor.

The corneal implant that is configured to correct irregularities of the curvature of the cornea of a subject has a dome-shaped structural body designed to impose a predefined curvature upon the corneal portions that are to be in contact with the implant. This structural body comprises:
an outer peripheral ring having a central axis, the outer peripheral ring lying in a first plane;
an inner peripheral ring set around the central axis in a second plane offset with respect to the first plane along the central axis; and
a connection structure pre-arranged to connect the outer peripheral ring and the inner peripheral ring, the connection structure including at least one connection element that extends along a path connecting the outer peripheral ring to the inner peripheral ring,
the connection element having, in a projection plane parallel to the first plane or the second plane, a profile comprising at least one curvilinear stretch.

A further embodiment of the disclosure comprises a precursor for corneal implant, which comprises structural body, the structural body comprising:
an outer peripheral ring;
an inner peripheral ring; and
a connection structure pre-arranged to connect the outer peripheral ring and the inner peripheral ring,
wherein said outer peripheral ring, said inner peripheral ring, and said connection structure lie in one and the same plane, and
wherein said outer peripheral ring and said inner peripheral ring lie at a first distance apart,
said connection structure being designed to undergo a deformation to obtain a predefined dome-like conformation of said structural body wherein said outer peripheral ring lies in a first plane and said inner peripheral ring lies in a second plane distinct from and parallel to said first plane and at a second distance from said outer peripheral ring greater than said first distance,
wherein the connection structure comprises at least one connection element for connecting the outer peripheral ring to the inner peripheral ring that extends along a profile deviating from a connection path of minimum length, equal to said first distance, so as to have a length in excess of said first distance that is such as to cover at least said second distance between said outer peripheral ring and said inner peripheral ring in said predefined dome-like conformation of said structural body.

A further embodiment regards a corneal implant configured to correct irregularities of the corneal curvature obtained from the precursor through a process of forming of the structural body. In particular, the implant may be obtained by means of deformation of the connection structure such that the outer peripheral ring lies in a first plane and the inner peripheral ring lies in a second plane distinct from and parallel to the first plane, and said structural body as a whole assumes a dome-like conformation.

### BRIEF DESCRIPTION OF THE DRAWINGS

The invention will now be described in detail, purely by way of illustrative and non-limiting example, with reference to the annexed drawings, wherein:

FIG. 1 is a perspective view of a corneal implant positioned in contact with an eyeball according to an embodiment that forms the subject of the present disclosure;

FIG. 2 is a cross-sectional view of embodiments of the corneal implant that forms the subject of the present disclosure;

FIG. 3 illustrates a precursor of a corneal implant according to an embodiment forming the subject of the present disclosure;

FIG. 4 illustrates a precursor of a corneal implant according to an embodiment forming the subject of the present disclosure;

FIG. 5 illustrates a precursor of a corneal implant according to an embodiment forming the subject of the present disclosure;

FIG. 6 illustrates a precursor of a corneal implant according to an embodiment forming the subject of the present disclosure;

FIG. 7 illustrates a precursor of a corneal implant according to an embodiment forming the subject of the present disclosure;

FIG. 8 illustrates a precursor of a corneal implant according to an embodiment forming the subject of the present disclosure;

FIG. 9 illustrates a precursor of a corneal implant according to an embodiment forming the subject of the present disclosure;

FIG. 10 illustrates a precursor of a corneal implant according to an embodiment forming the subject of the present disclosure;

FIG. 11 illustrates a precursor of a corneal implant according to an embodiment forming the subject of the present disclosure;

FIG. 12 illustrates a precursor of a corneal implant according to an embodiment forming the subject of the present disclosure;

FIG. 13 illustrates a precursor of a corneal implant according to an embodiment forming the subject of the present disclosure;

FIG. 14 illustrates a precursor of a corneal implant according to an embodiment of the corneal implant forming the subject of the present disclosure; and

FIG. 15 illustrates a precursor of a corneal implant of an embodiment of the corneal implant forming the subject of the present disclosure.

### DETAILED DESCRIPTION OF THE INVENTION

The invention will now be described in detail, purely by way of illustrative and non-limiting example.

In the ensuing description, numerous specific details are presented to enable a complete understanding of the embodiments. The embodiments may be implemented without one or more of the specific details, or with other methods, components, materials, etc. In other cases, well-known structures, materials, or operations are not illustrated or described in detail so that certain aspects of the embodiments will not be obscured.

Throughout the present description, reference to “an embodiment” or “one embodiment” means that a particular detail, structure, or characteristic described in connection with the embodiment is included in at least one embodiment. Hence, phrases such as “in a certain embodiment” or “in one embodiment” that may appear in various points throughout the present description do not necessarily always refer to one and the same embodiment. Moreover, the particular details, structures, or characteristics may be combined in any suitable way in one or more embodiments.

The headings provided herein are for convenience only and do not interpret the scope or meaning of the embodiments.

The corneal implant forming the subject of the present disclosure is an implant with dome-shaped structural body that comprises an outer peripheral ring, an inner peripheral ring, and a connection structure comprised between the two rings. This implant is designed for the treatment of patients affected by an irregular conformation of the cornea in so far as it is able to impose its own curvature upon the cornea, thus correcting pathological de formations thereof thanks to the stiffness of its own structure and to the presence of one or more connection elements between the outer peripheral ring and the inner peripheral ring; such connection elements, have, in a projection plane, such as the plane in which the outer peripheral ring lies, at least one curvilinear profile. This characteristic presents the advantage that, during the operation of drawing from a two-dimensional configuration of the implant (hereinafter “precursor”) to a three-dimensional configuration of the implant, the one or more connection elements can cover an increased distance between the outer peripheral ring and the inner peripheral ring. Such connection elements may have a length equal to or greater than the distance between the outer peripheral ring and the inner peripheral ring after forming thereof. Such connection elements may consequently follow the shape of the mould that has the desired curvature without causing undesirable rupture of the structure of the implant.

The above characteristic makes it possible to achieve a series of advantages over known corneal implants, such as the implants described in the document EP 3 801 386 A1, which present a structural body with one or more series of connection elements between the outer peripheral ring and the inner peripheral ring, which—in plan view, in a projection plane in which the outer peripheral ring lies—have only rectilinear profiles.

The implant forming the subject of the present disclosure has been designed to present a wide area of its surface that is substantially free or empty in order not to interfere at all or at most interfere only minimally with entry of light into the eye.

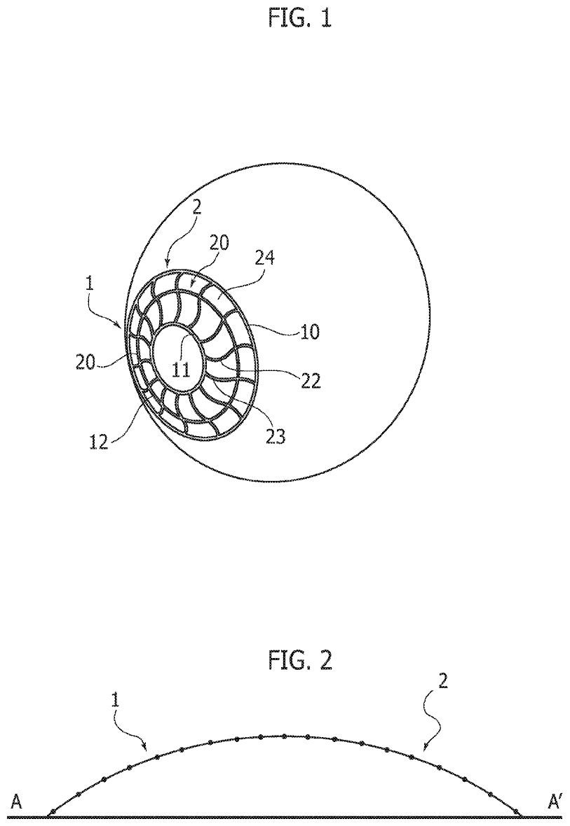

The implant described, comprising a dome-shaped structural body, as illustrated, for example, in FIGS. 1 and 2 and designated by the reference number 2, may derive from a two-dimensional precursor 100 subjected to deformation, preferably subjected to forming, more preferably subjected to thermoforming. Advantageously, the precursor may be subjected to an operation of drawing and subsequent coining with a mould formed by a die and a punch. The subject of one or more embodiments of the present disclosure is hence also a precursor—for example designated in FIGS. 3 to 15—where the different structural elements lie in one and the same plane. Once subjected to forming, the precursor assumes a three-dimensional dome-like configuration, as illustrated, for example, in FIG. 1 and designated by the reference number 2. The dome-shaped structural body 2 may have a curvature such as to give rise in sagittal section to a substantially semicircular profile, as illustrated in FIG. 2, or a semi-elliptical profile, or else a profile predefined on the basis of the needs of the subject.

The structural body 2 of the implant 1, as likewise of the precursor 100, comprises an outer peripheral ring 10 with a central axis and lies in a first plane (denoted by AA′ in FIG. 2). The structural body 2 further comprises an inner peripheral ring 11 set around the central axis and lying in a second plane offset with respect to the first plane along the central axis. Preferably, the outer peripheral ring 10 and the inner peripheral ring 11 are coaxial.

The structural body 2 further comprises a connection structure 20 that is pre-arranged to connect the outer peripheral ring 10 and the inner peripheral ring 11. This connection structure 20 comprises at least one connection element 23 that extends along a path connecting the outer peripheral ring 10 to the inner peripheral ring.

The connection structure 20 comprises empty portions (or meshes) 24. The total area of the empty portions 24 within the connection structure 20 is comprised between 40% and 99.9% of the surface of the internal structure.

Moreover, the contour of the empty portions 24 may present only non-rectilinear profiles that do not extend over straight-line segments. The contour of the empty portions 24 may only have undulated contours.

With reference to the precursor 100, the outer peripheral ring 10, the inner peripheral ring 11, and the connection structure 20 lie in one and the same plane. Preferably, the outer peripheral ring 10 and the inner peripheral ring 11 are coaxial. Moreover, the outer peripheral ring 10 and the inner peripheral ring 11 lie in one and the same plane at a first distance apart (denoted, for example in FIG. 3, by the letter D). The connection structure 20 is to undergo deformation to obtain a predefined dome-like conformation of the structural body 2. Once the dome-like conformation of the structural body 2 has been obtained, the outer peripheral ring 10 lies in a first plane and the inner peripheral ring 11 lies in a second plane distinct from and parallel to the first; in particular, the outer peripheral ring 10 and the inner peripheral ring 11 are located at a second distance apart that will be greater than the first distance. The connection structure 20 presents the characteristic of comprising at least one connection element 23 for connecting the outer peripheral ring 10 to the inner peripheral ring 11 that extends along a profile 22 deviating from a connection path of minimum length, equal to said first distance: in particular, the connection element 23 has a length in excess of the first distance in such a way as to be able to cover the second distance between the outer peripheral ring 10 and the inner peripheral ring 11 in the predefined dome-like conformation of the structural body 2, without undergoing failure or any uncontrolled deformation.

In other words, the one or more connection elements 23 of the implant 1 may have a length greater than the minimum distance (understood as length of the stretch of straight line that joins the two rings and that corresponds to the minimum path between them) between the outer peripheral ring 10 and the inner peripheral ring 11 when the aforesaid rings lie in one and the same plane (two-dimensional configuration of the implant, or precursor of the implant). In particular, as illustrated in the figures, the connection structure 23 comprises one or more connection elements 23 that have a profile 22 comprising at least one curvilinear stretch, preferably a meandering or falciform stretch. s By way of example, the distance (first distance) between the outer peripheral ring 10 and the inner peripheral ring 11 that lie in one and the same plane (referred to, in the present disclosure, as first distance) may be comprised between 0.01 mm and 10 mm, for example 2.50 mm. The connection element 23 deviating from a connection path of minimum length may have a length comprised between 0.02 mm and 20 mm, for example 2.60 mm.

Following upon deformation induced by forming, the aforesaid one or more connection elements 23, given that they preferably have a profile with at least one curvilinear stretch, can extend SO as to cover an increased distance between the outer peripheral ring and the inner peripheral ring, without undergoing phenomena of failure or uncontrolled deformation. As illustrated, for example, in FIG. 1, the one or more connection elements 23 of the connection structure 20 that has assumed a dome-like conformation have a profile 22 that, even after forming, comprises at least one curvilinear stretch, preferably a meandering or falciform stretch.

In one or more embodiments, the connection structure 20 of the precursor 100, as likewise of the implant 1 deriving therefrom, may comprise a plurality of connection elements 23 or else a single connection element 23.

In the case of a plurality of connection elements 23, they may have an irregular distribution in the connection structure 20 or else a uniform distribution. The connection elements 23 may be located close to one another in one area (for example, corresponding to the area of the cornea where the keratoconus is located) and sparser in another area (for example, corresponding to an area of the cornea without keratoconus).

In both cases, the one or more connection elements 23 may comprise a first end 31 connected to the outer peripheral ring 10 and a second end 32 connected to the inner peripheral ring 11. Each element of the plurality of connection elements 23 or the single connection element 23 extends along a path connecting the outer peripheral ring 10 to the inner peripheral ring 11 and has a profile comprising at least one curvilinear stretch.

In one or more embodiments, the plurality of connection elements 23 may have profiles that define one or more corresponding cavities oriented in one and the same direction of rotation with respect to the central axis. Examples of such embodiments are illustrated, for instance, in FIGS. 1, 3-7, and 10-13.

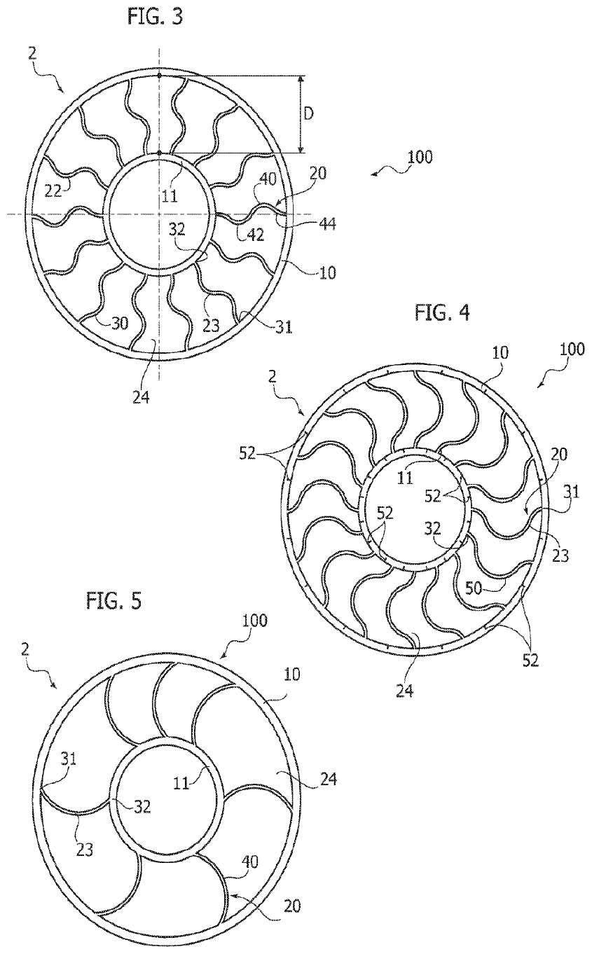

When the connection structure 20 has a plurality of connection elements 23, they may have different profiles. The profile may be sinusoidal 30 (FIGS. 1, 3, 10, 11), arched 40 (FIGS. 5-7, 13), omega-shaped 50 (FIGS. 4, 12), ring-shaped 60 (FIG. 8), or else may present a combination of the aforesaid shapes.

In the case of the sinusoidal profile, illustrated, for example, for the implant of FIG. 1 or for the precursor 100 of FIG. 3, it may comprise points of maximum 40 and points of minimum 42 that project circumferentially on opposite sides with respect to zero points 44.

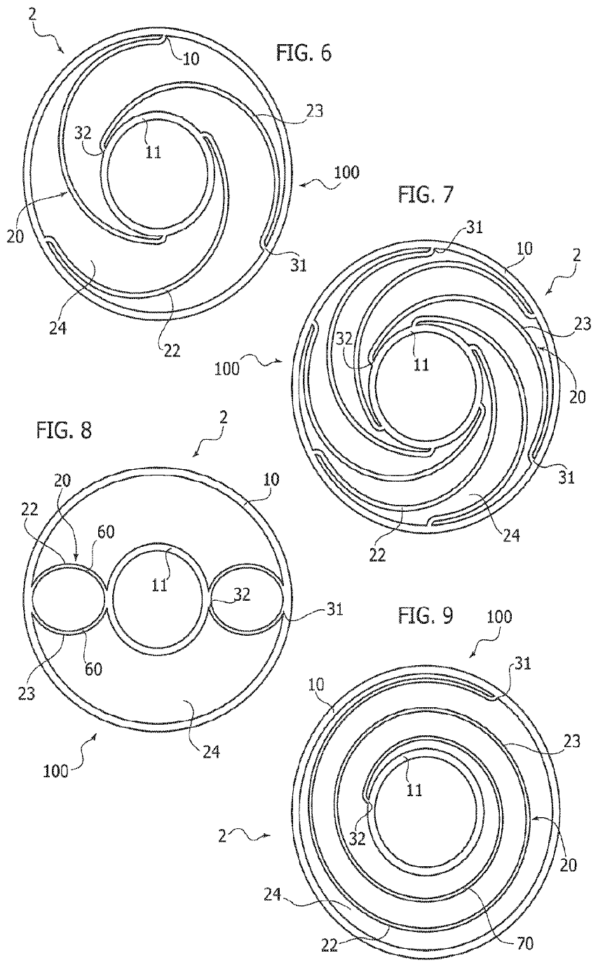

An example of structural body 20 comprising at least one connection element 23 with omega-shaped profile is represented in FIG. 4. FIGS. 5 to 7 illustrate precursors of a corneal implant 1, where the connection element 23 has an arched profile. In particular, FIG. 5 illustrates a structural body 2 comprising seven connection elements 23 with arched profile. In this example, the connection elements 23 are distributed in an asymmetrical and irregular way within the connection structure 20. FIGS. 6 and 7 illustrate precursors with a different number of connection elements 23 that each define a semicircumference. This semicircumference may have a diameter larger than the diameter of the inner peripheral ring 11.

In one or more embodiments, the first end 31 and the second end 32 of a first connection element 23 with arched profile may be adjacent to a respective first end 31 and a respective second end 32 of a second, arched, connection element 23 to form an inner ring 60 comprised in the connection structure 20 between the outer peripheral ring 10 and the inner peripheral ring 11. The precursor 100, as likewise the implant deriving therefrom, may comprise at least two pairs of elements 23 with arched profile facing one another to form at least two inner rings 60 (FIG. 8).

FIG. 9 illustrates a precursor 100 in which the connection structure 20 comprises a single connection element 23, which comprises a first end 31 connected to the outer peripheral ring 10 and a second end 32 connected to the inner peripheral ring 11. In this embodiment, the connection element 23 extends along a path connecting the outer peripheral ring to the inner peripheral ring. Preferably, the connection element 23 develops according to a spiral path 70.

In order to bestow greater structural stability upon the implant, the precursor 100, as likewise the implant deriving therefrom, may comprise in the connection structure 20 at least one bridging structure 26. Said structure 26 connects adjacent elements of a plurality of connection elements 23 (as illustrated in FIGS. 10 to 13) or else at least two distinct and separate points of the single connection element 23 (as illustrated in FIGS. 14 and 15). The above bridging structures 26 can bestow structural stability upon the implant. The above bridging structures 26 may have a curvilinear profile.

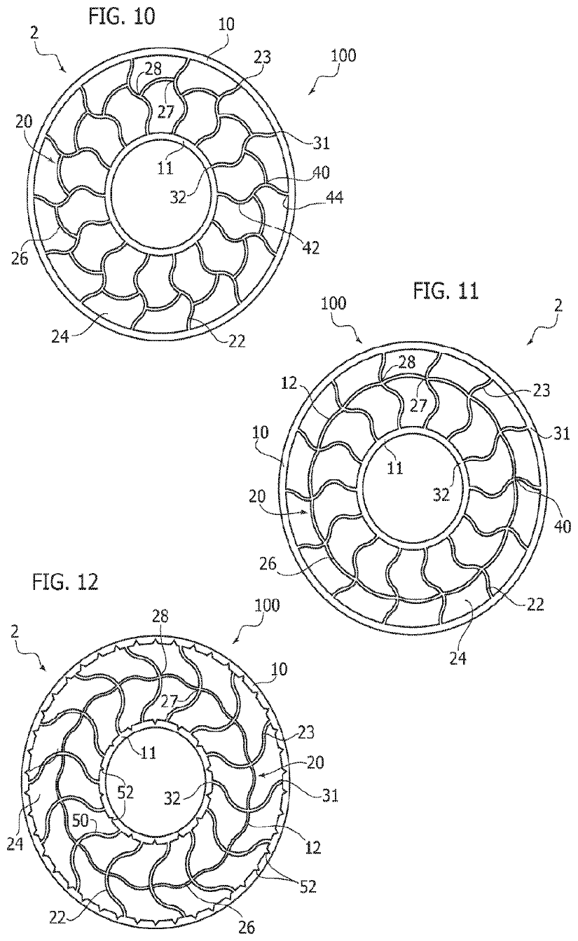

With reference to the precursor illustrated in FIG. 10, the first end 27 of one bridging structure 26 is connected at a point of maximum or a point of minimum of a connection element 23 with sinusoidal profile and the second end 28 of the bridging structure 26 is connected at a zero point of at least one connection element 23 with sinusoidal profile that is consecutive in a circumferential direction. Alternatively, as illustrated in FIG. 11, the first end 27 of a bridging structure 26 can be connected at a point of maximum 40 or a point of minimum 42 of a connection element 23 with sinusoidal profile, and the second end 28 of the bridging structure 26 is connected, respectively, at a point of maximum 40 or a point of minimum 42 of a connection element 23 with sinusoidal profile that is consecutive in a circumferential direction.

FIG. 11 illustrates an example in which bridging structures 26 connect omega-shaped connection elements 23.

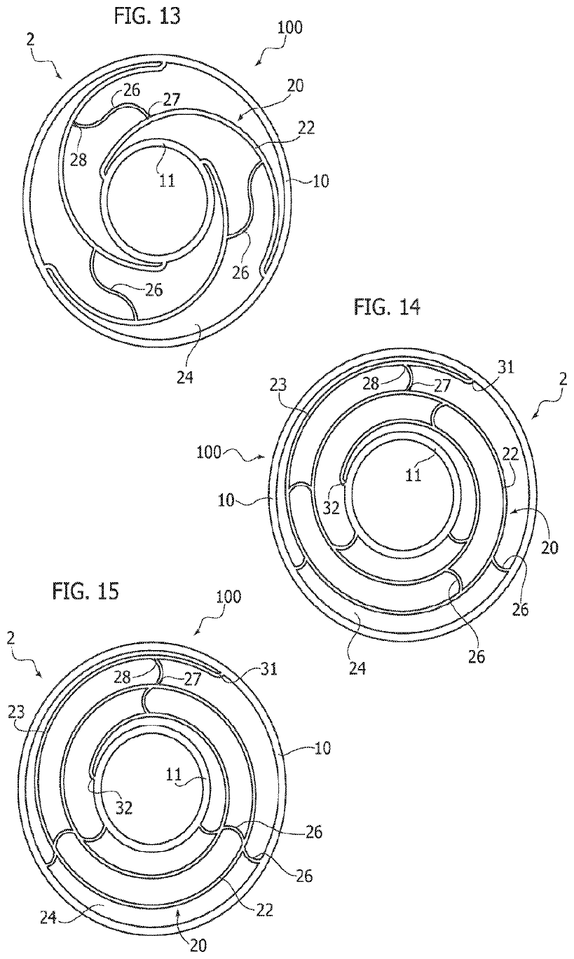

The bridging structures 26 may be arranged according to an annular arrangement to form at least one ring 12 that is intermediate between the outer peripheral ring 10 and the inner peripheral ring 11 and is coaxial therewith, as illustrated, for example, in FIGS. 11 and 12. In FIG. 13 the bridging structures 26 connect two consecutive arched connection elements 23 and have a curvilinear profile. FIGS. 14 and 15 illustrate two examples in which the bridging structures 26 are distributed between turns of the connection element 23 with spiral profile.

In one or more embodiments, at least one between the outer peripheral ring 10 and the inner peripheral ring 11 of the implant 1 and/or of the precursor 100 may have weakening points 52 (preferably notches), comprising slits facing the inside of the connection structure 20, visible, for example, in FIG. 4 and in FIG. 12.

As described, the connection elements 23 of the precursor 100 extend along a profile 22 that deviates from a connection path of minimum length between the outer peripheral ring and the inner peripheral ring when they lie in one and the same plane. The characteristic whereby the connection element 23 has a length in excess that makes it possible to cover at least the distance between the outer peripheral ring 10 and the inner peripheral ring 11 once the dome-like deformation of the structural body has been obtained presents advantages during deformation. To favour this characteristic, the connection element may have a profile with at least one curvilinear stretch. In particular, even after deformation, the one or more connection elements 23, in plan view, in the projection plane in which the outer peripheral ring lies, do not have a rectilinear profile, but a curvilinear one.

The above characteristics present the advantage that, during the operation of drawing from a two-dimensional configuration of the precursor to a three-dimensional configuration of the implant, the connection elements 23 can cover an increased distance between the outer peripheral ring and the inner peripheral ring; these elements 23 can consequently take on the shape of the mould that has the desired curvature. This enables the structural body 20 to spread out and assume the appropriate curvature; the desired curvature may form an ellipsoid, the semi-axes of which may have, for example, a size comprised between 7 mm and 11 mm.

Known precursors for corneal implants comprising rectilinear connection elements do not favour an adequate lengthening during forming and consequently prove less suited to the purpose. In particular, forming of such connection elements may lead to single or multiple failures as well as anomalous, undesired, and uncontrollable deformations of the connection elements themselves but also, in certain cases, of the outer peripheral ring and/or the inner peripheral ring.

Also the presence of weakening points, for example notches, at the level of the outer peripheral ring and/or the inner peripheral ring contributes to favouring a correct forming (or thermoforming) and drawing during the process of production of the implant. These weakening points may contribute to preventing single and/or multiple failures of the one or more connection elements themselves or else to preventing anomalous, undesired and uncontrollable deformations of the three-dimensional configuration of the precursor after (thermo) forming or else of the inner ring or the outer ring. These weakening points may moreover contribute to preventing single and multiple failures of the inner ring and/or the outer ring or other connection structures.

The implant forming the subject of the present disclosure may also be produced, for example, by means of three-dimensional (3D) printing. The present applicants has noted that, irrespective of the process used for its production, an implant provided with a connection structure 20 such as to reproduce the profiles of FIGS. 3 to 15 in a projection plane parallel to the planes of the two rings presents advantages in terms of an effective correction of irregularities of the curvature of the cornea of a subject since it has a curvature that follows precisely the curvature of the healthy human cornea. The presence of connection elements 23 with curvilinear profiles enhances the efficacy of the implant in obtaining a predefined curvature of the cornea. The absence of rectilinear profiles in (and between) the connection elements defines a connection structure 20 without sharp corners between the various elements. This characteristic contributes to countering onset of fibrotic phenomena due to non-rounded corners (especially in the proximity of right-angle corners of the implant once it has been implanted and during implantation in the cornea of a subject.

In the ensuing description of the implant, FIGS. 3 to 15 are in this case to be understood as representing the projections of the implant in question in a projection plane parallel to the first plane or the second plane, for example in the plane in which the outer peripheral ring lies.

The corneal implant 1 has a dome-shaped structural body 2 designed to impose a predefined curvature upon the corneal portions that are to be in contact with the implant. The structural body 2 comprises:
an outer peripheral ring 10 having a central axis, the outer peripheral ring 10 lying in a first plane;
an inner peripheral ring 11 set around the central axis in a second plane offset with respect to the first plane along the central axis; and
a connection structure 20 pre-arranged to connect the outer peripheral ring 10 and the inner peripheral ring 11. The connection structure 20 comprises at least one connection element 23 that extends along a path connecting the outer peripheral ring 10 to the inner peripheral ring 11, the connection element 23 having in a projection plane parallel to the first plane or the second plane, a profile 22 comprising at least one curvilinear stretch, preferably a meandering or falciform stretch.

The connection structure comprises empty portions (or meshes) 24. The total area of the empty portions 24 within the connection structure 20 is comprised between 40% and 99.9% of the surface of the internal structure.

Moreover, the contour of the empty portions 24 may only have non-rectilinear profiles that do not extend over straight-line segments. The contour of the empty portions 24 may only have undulated contours.

In one or more embodiments, the connection structure 20 may comprise a plurality of connection elements 23 or else a single connection element 23.

In the case of a plurality of connection elements 23, they may have an irregular distribution in the connection structure 20 or else a uniform distribution. The connection elements 23 may be located close to one another in one area (for example, corresponding to the area of the cornea in which the keratoconus is located or which requires corrections) and sparser in another area (for example, corresponding to an area of the cornea that is without keratoconus or that does not require corrections).

In either case, the one or more connection elements 23 may comprise a first end 31 connected to the outer peripheral ring 10 and a second end 32 connected to the inner peripheral ring 11. Each element of the plurality of connection elements 23 or the single connection element 23 extends along a path connecting the outer peripheral ring to the inner peripheral ring and has a profile comprising at least one curvilinear stretch.

In one or more embodiments, the plurality of connection elements 23 may have profiles that define one or more corresponding cavities oriented in one and the same direction of rotation with respect to the central axis. Examples of such embodiments are illustrated, for example, in FIGS. 1, 3-7, and 10-13.

When the connection structure 20 has a plurality of connection elements 23, they may have different profiles. The profile may be meandering, for example sinusoidal 30 (FIGS. 1, 3, 10, and 11), or else falciform, for example arched 40 (FIGS. 5-7 and 13), omega-shaped 50 (FIGS. 4 and 12), or ring-shaped 60 (FIG. 8), or else may present a combination of the aforesaid profiles.

In the case of the sinusoidal profile, illustrated, for example, for the implant of FIG. 1, it may comprise points of maximum 40 and points of minimum 42 projecting circumferentially on opposite sides with respect to zero points 44.

An example of structural body 20 comprising at least one connection element 23 with omega-shaped profile is represented in FIG. 4. FIGS. 5 to 7 illustrate connection elements 23 with arched profile. In particular, FIG. 5 illustrates a structural body 2 comprising seven connection elements 23 with arched profile. In this example, the connection elements 23 are distributed in an asymmetrical and irregular way within the connection structure 20. FIGS. 6 and 7 illustrate a different number of connection elements 23 that define a semicircumference. Such a semicircumference may have a diameter larger than the diameter of the inner peripheral ring 11.

In one or more embodiments, the first end 31 and the second end 32 of a first connection element 23 with arched profile may be adjacent to a respective first end 31 and a respective second end 32 of a second, arched, connection element 23 to form an inner ring 60 comprised in the connection structure 20 between the outer peripheral ring 10 and the inner peripheral ring 11. The implant may comprise at least two pairs of elements 23 with arched profile facing one another to form at least two inner rings 60 (FIG. 8).

The connection structure 20 illustrated in FIG. 9 comprises a single connection element 23, comprising a first end 31 connected to the outer peripheral ring 10 and a second end 32 connected to the inner peripheral ring 11. In this embodiment, the connection element 23 extends along a path connecting the outer peripheral ring to the inner peripheral ring. Preferably, the connection element 23 develops according to a spiral path 70.

In order to bestow a greater structural stability upon the implant, the latter may comprise in the connection structure 20 at least one bridging structure 26. This structure 26 connects adjacent elements of a plurality of connection elements 23 or else at least two distinct and separate points of the single connection element 23. The aforesaid bridging structures 26 have two ends 27, 28 connected to adjacent elements of a plurality of connection elements 23, or else to at least two distinct and separate points of the single connection element 23. The bridging structures may bestow structural stability upon the implant 1. These bridging structures 26 may have a curvilinear profile.

For instance, with reference to FIG. 10, the first end 27 of one bridging structure 26 is connected at a point of maximum or a point of minimum of a connection element 23 with sinusoidal profile and the second end 28 of the bridging structure 26 is connected at a zero point of at least one connection element 23 with sinusoidal profile that is consecutive in a circumferential direction. Alternatively, as illustrated in FIG. 11, the first end 27 of one bridging structure 26 may be connected at a point of maximum 40 or a point of minimum 42 of a connection element 23 with sinusoidal profile, and the second end 28 of the bridging structure 26 is connected, respectively, at a point of maximum 40 or a point of minimum 42 of a connection element 23 with sinusoidal profile that is consecutive in a circumferential direction. The reticular structure comprises empty portions formed between bridging structures and connection elements that preferably have contours without profiles having sharp corners that might be the cause of fibrosis once the device is implanted in vivo in a cornea.

FIG. 11 illustrates an example in which bridging structures 26 connect omega-shaped connection elements 23.

The bridging structures 26 may be arranged according to an annular arrangement to form at least one ring 12 intermediate between the outer peripheral ring 10 and the inner peripheral ring 11 and coaxial with them, as illustrated, for example, in FIGS. 11 and 12.

In FIG. 13 the bridging structures 26 connect two consecutive arched connection elements 23 and have a curvilinear profile.

FIGS. 14 and 15 illustrate two examples in which the bridging structures 26 are distributed between turns of the connection element 23 with spiral profile.

In one or more embodiments, at least one between the outer peripheral ring 10 and the inner peripheral ring 11 of the implant may have weakening points 52 (preferably notches), comprising slits facing the inside of the connection structure 20, visible, for example, in FIG. 4 and in FIG. 12.

The precursor, as likewise the corneal implant forming the subject of the present disclosure, may have the outer peripheral ring 10 with a preferably circular shape, the diameter of which is comprised between 1 mm and 20 mm, preferably between 5 mm and 10 mm. The inner peripheral ring 10 may preferably have a circular shape, the diameter of which may be comprised between 0.1 mm and 10 mm, preferably between 0.5 mm and 7 mm.

The outer peripheral ring 10 and/or the inner peripheral ring 11 may have a dimension of the cross section in the plane of the peripheral rings 10, 11, i.e., a width, preferably ranging from 10 μm to 1000 μm, more preferably comprised between 15 μm and 250 μm.

The connection elements 23 and the bridging structures 26 preferably have a width ranging from 10 to 250 μm, more preferably greater than 30 μm and less than 150 μm.

The outer peripheral ring 10, the inner peripheral ring 11, and the connection elements 23 and the bridging structures 26 have a dimension of the aforesaid section in a direction perpendicular to the width or to the plane of the peripheral rings 10, 11, i.e., a thickness, ranging from 5 μm to 250 μm, more preferably greater than 10 μm and less than 100 μm.

If the cross section of the aforesaid rings and/or elements is circular or elliptical, the above two dimensions will be substantially the same.

The empty portions 24 within the meshes of the connection structure 20 preferably each have an area comprised between 0.5 mm² and 2.5mm².

The total surface of the meshes preferably has an area comprised between 2 mm² and 40 mm².

The total volume of the mesh is preferably comprised between 0.10 mm³ and 0.80 mm³.

The total weight of the mesh is preferably comprised between 0.001 g and 0.004 g.

The corneal implant forming the subject of the present disclosure presents mechanical and structural characteristics, in particular a stiffness higher than the stiffness of the corneal tissue, such as to impose its own curvature upon the cornea. This structural characteristic of the implant makes it possible, following upon surgical grafting thereof in the thickness of the corneal stroma or under the corneal epithelium, to restore an optimal optical situation by rendering of regular shape corneas having deformations such as to generate optical aberrations that jeopardize, for example, visual acuity or sensitivity to contrast.

In one or more embodiments, the precursor and/or the implant may be made of a material selected from among: metals and corresponding alloys, for example grade-1 or grade-2 titanium, nickel, cobalt, chromium, tantalum, gold, silver, iron and their alloys, such as steel, Nitinol, Ti₆Al₄V, AISI 301®; carbon and its compounds, preferably inorganic ones; polymers; ceramic materials; combinations thereof; superelastic materials, preferably cobalt-chromium alloys (Phynox); and stainless steel without nickel and cobalt (Vasculoy).

The stiffness of the human cornea, evaluated through the measurement of Young's modulus, is found to assume values in the range of from 0.01 to 50 MPa, preferably from 0.1 to 30 MPa.

In general, the stiffness of the corneal implant forming the subject of the present disclosure is preferably greater than 1 MPa, more preferably greater than 30 MPa, even more preferably comprised in a range of from 50 MPa to 300 GPa.

The device the other hand enables, by virtue of its stiffness, reduction of the optical aberrations generated both by the anterior corneal surface and by the posterior corneal surface in the case where the implant is inserted into the corneal stroma (where “anterior” and “posterior” are to be understood with respect to the position of insertion of the corneal implant into the cornea), a correction that cannot be achieved with ablations by means of excimer laser or femtosecond laser or with ring-shaped corneal implants such as ICRS or again implants made of non-rigid materials, such as a fabric, or else again implants made with connection elements or bridging structures that do not bestow also structural properties upon the implant. In the case where the implant is positioned underneath the corneal epithelium, the latter by regrowing will come to recover and incorporate the implant, assuming the shape thereof, smoothing and levelling any possible irregularities induced on the corneal tissues by the presence or by the geometry of the mesh, and filling the voids present in the mesh.

Furthermore, following upon surgical treatment that may envisage dissection of the cornea, since the corneal tissue has a lower structural strength as compared to the pre-cut corneal tissue, it will adapt better to the new geometry imposed by the corneal implant with quantitatively superior optical results. In other words, the cornea within which the corneal implant has been inserted will have a structural stiffness suitable for proper maintenance over time of the optical correction imposed by the corneal implant.

On the other hand, in view of the rigidity of the corneal implant described herein, the implant is able to preserve its own dome-like configuration even following upon stresses exerted during implantation (for example, on account of handling of the implant by the surgeon) or during daily use (for example, on account of the patient rubbing his or her eyes).

The device is indicated in all pathological conditions that may lead to an irregularity of the corneal curvature—preferably provided that the stroma has sufficient transparency—that is such as to generate optical aberrations that cannot be corrected, for example, with spectacles or contact lenses, or in subjects that do not tolerate contact lenses.

In addition, the device forming the subject of the present disclosure may be implanted in order to change the corneal curvature in healthy subjects to correct a refractive error.

The main categories of patients at which the device is aimed comprise subjects affected by non-inflammatory ectasia of the cornea (as, for example, keratoconus and pellucid marginal degeneration or else ectasia following upon procedures of corneal refractive surgery) and subjects who have undergone deep anterior penetrating or lamellar keratoplasty and who present irregular or marked astigmatism.

The use of the corneal implant forming the subject of the present disclosure makes it possible to achieve moreover the following benefits. Thanks to the mechanical stiffness of the reticular structure, the corneal implant enables modelling of the cornea by imposing and obtaining a curvature defined beforehand, contrary to what occurs with the use of ICRS, and without ablation of tissue, as occurs in the case of ablation treatments with excimer laser or femtosecond laser in which the final conformation of the cornea is not foreseeable exactly, and the treatment is irreversible. On the other hand, thanks to the wide area of the empty portions 24 of the meshes of the reticular structure as compared to the total area of the reticular structure itself, the corneal implant forming the subject of the present disclosure i) does not interfere (or at most interferes to an altogether negligible extent) with passage of oxygen and other molecules through the anterior and posterior corneal tissue to the implant and ii) leads to a reduction of the incoming light by 5-10% and to altogether negligible diffractive phenomena if compared to the benefits achieved by the implant 1 in terms of reduction of low-order aberrations (astigmatism) and high-order aberrations (such as coma and trefoil aberrations). Thanks to positioning of the corneal implant in the proximity of the nodal point of the eye, the implant itself is substantially not perceived by the patient.

The corneal implant forming the subject of the present disclosure can preferably be implanted in the corneal stroma or fixed to the anterior surface of the corneal stroma, underneath the corneal epithelium. The implant can on the other hand be easily grafted and removed without damage or with minimal damage to the corneal tissue, considerably reducing the pain in the postoperative period, speeding up visual recovery and thus improving the quality of life of the patient.

The implant forming the subject of the present disclosure presents evident advantages in terms of an effective correction of irregularities of the curvature of the cornea of a subject in so far as it has a curvature that follows precisely the curvature of the healthy human cornea, and, for example during the production process of forming, does not undergo: i) single and/or multiple ruptures of the connection elements, and/or of the inner ring and/or the outer ring; ii) anomalous deformations; and iii) undesired and uncontrollable deformation of the three-dimensional configuration.

The dimensions of the corneal implant 1 understood as diameter of the outer peripheral ring 10, diameter of the inner peripheral ring 11, radius of curvature of the dome-shaped structural body 2, as well as the thickness of the peripheral rings 10, 11 and/or of the elements 23 and 26 will be selected in such a way as to adapt to the specific needs of the patient and to the morphology of the cornea.

### Modes of Grafting of the Corneal Implant

The implant forming the subject of the present disclosure can be grafted in the cornea of a patient by means of a surgical operation of a conservative-additive refractive type that does not envisage subtraction of corneal tissue.

The device may be preferably implanted in the corneal stroma, but may also be fixed to the anterior surface of the corneal stroma, underneath the corneal epithelium or Bowman's membrane.

In the case of grafting within the corneal stroma, there is formed in the stroma a pocket of a circular shape having a diameter preferably of from 6 mm to 12 mm, preferably using a femtosecond laser at a depth quantifiable in a range of from 10 μm to 700 μm, preferably between 70 and 400 μm, with respect to the anterior corneal surface, or else is prepared using a microkeratome or a femtosecond laser, and a side cut having a length of from 2 mm to 5 mm, into which the device is inserted once it has been folded on itself. Alternatively, if the material of which the implant is made does not allow the latter to be folded on itself, it is also possible to provide a flap having a diameter quantifiable in a range of from 3 mm to 11 mm, preferably between 5 and 9 mm, and a thickness of from 50 μm to 500 μm, preferably between 70 and 400 μm.

It is possible for the free margin of the flap, or of the layer in front of the pocket, or of the side cut to be fixed to the adjacent tissue with stitches, or else metal clips, or else fibrin glues.

It is moreover possible to obtain in the corneal stroma a negative having a shape exactly congruent with the corneal implant by means of ablation of tissue with excimer laser or femtosecond laser, into which the corneal implant itself is then to be inserted.

### Materials That can be Used for Producing the Corneal Implant

The corneal implant may be obtained using: metals and corresponding alloys (by way of example, there may be cited titanium, such as grade-1 or grade-2 titanium, nickel, cobalt, chromium, tantalum, gold, silver, iron and their alloys, such as steel, Nitinol, Ti₆Al₄V, AISI 301®, etc.), the metals being preferably non-magnetic; carbon and its compounds, preferably inorganic ones; polymers; ceramic materials; and combinations thereof. Further materials that can be employed may be selected between superelastic materials, such as cobalt-chromium alloys (Phynox), and stainless steel without nickel and cobalt (Vasculoy).

The corneal implant 100 may likewise be obtained using the aforesaid materials further mixed with other compounds such as, by way of example, hydroxyapatite, polylactic acid, polycaprolactone, fibroin, chitin, cellulose, chitosan, gelatin, carboxymethyl cellulose, (human or animal) collagen, hydrocolloid, hydrogel, Crabyon®, silver.

The corneal implant 100 may moreover be provided with an outer coating or filling in the voids that is biocompatible and/or biodegradable, optionally comprising a pharmacologically active principle, preferably anti-inflammatory, antibacterial, and/or designed to inhibit or control the fibrotic reaction around the implant, or else an outer coating or filling in the voids that will be active and capable of inducing regeneration of the damaged corneal tissues, such as somatic cells from donors differentiated into corneal epithelial cells and endothelial cells or else iPS (induced Pluripotent Stem) cells from healthy donors differentiated into corneal limbal stem cells and corneal endothelial cells using consolidated methods.

The outer coating may be obtained using compounds and/or compositions known in the sector of implantable prostheses in an animal body for the release of active principles by implantable devices/prostheses. Materials particularly preferred for producing a biocompatible and/or biodegradable coating of the corneal implant are selected from the following: hydroxyapatite, polylactic acid, polycaprolactone, fibroin, chitin, cellulose, chitosan, gelatin, carboxymethyl cellulose, (human or animal) collagen, hydrocolloid, hydrogel, Crabyon®, silver, and combinations thereof.

### Methods for Producing the Corneal Implant

Described in what follows, purely by way of non-limiting example, are some methods for producing a corneal implant as illustrated in FIG. 1.
A) Using as starting material, for example, a foil of commercially pure titanium (CP-Ti, ASTM B 265, Grade 2 having a thickness 0.05 mm—Lamina S.p.A.), the titanium foil is subjected to a cutting operation by laser machining in order to obtain the precursor 100.

Laser machining is carried out with the StarFemto FX laser with ultrashort-pulse source (Rofin Baasel Lasertech GmbH & Co. KG) with the following process parameters:
Wavelength: 1030 nm
Focussing Optics: F100
Galvo Scanner Head: S14
Scan Speed of Galvo Scanner Head: 200 mm/s
Field Size: 40 mm×40 mm
Pulse duration: 250 fs
Pulse energy: 20 μJ
Repetition rate: 20 kHz

Laser machining may also be performed using a StarFiber 180FC fibre source (Rofin Baasel Lasertech Gmbh & Co. KG). The result in terms of cutting precision is as good as the solution obtained with the StarFemto FX laser, but given the heat input there is a deformation and a quality of the cut wall that is poorer than that obtained with the StarFemto FX laser that mounts an ultrashort-pulse source.

Next, a chemical treatment of electropolishing is carried out according to techniques widely known in the art in order to clean the surface and remove undesired contaminants therefrom.

After electropolishing, the pieces can be subjected to a treatment of passivation/anodization if it is desired to introduce a change in colour of the piece.

The anodization treatment of the single pieces made of titanium is obtained according to the following procedure: the pieces are dipped in a basic aqueous solution with 10% ammonium sulphate, at room temperature for 10 s. The potential that is applied varies according to the desired colouring and, by way of non-exhaustive example, we provide the following indications on the potentials to be applied and the colours that can be obtained:
Dark brown: 12-15 V;
Purple: 35-40 V;
Light blue: 25-30 V;
Blue: 30 V;
Yellow: 40 V.

In order to bestow upon the connection structure 20 a generally dome-shaped configuration, the precursor is subjected to an operation of drawing and subsequent coining with a mould formed by a die and a punch. The die and the punch are made of tempered material (K100).

To produce prototypes of the corneal implant, it is preferable for the process of drawing and coining to be carried out using a mechanical press with precise laboratory mechanical toggle (GECHTER, 5HKPU), with a punch precision of +/−10 μm and a pressure value of 9 kg.

For small-series production, it is preferable for the process of drawing and coining to be carried out using a press with electro-actuated descent of the ram (ALFAMATIC, COLOMBO), with a punch precision of +/−10 μm and a pressure value of 9 kg.

At the end of the above step there may be envisaged a subsequent final cleaning (for example, in ultrasonic bath) to remove any possible further residue still present on the surface of the corneal implant at the end of the production process.

The corneal implant may be subjected also to one or more machining operations, laser operations, chemical operations, or operations of any other kind that have as aim modification of the surface morphology of the implant itself. Some possible machining operations are shot peening, corundum sandblasting, and passivation (which restores the maximum resistance to corrosion to the passive layer of oxide film, promoting formation thereof).
B) In the case where the starting material is constituted by an electrowelded mesh, this will be subjected to a cutting operation by laser machining in order to isolate the reticular structure 20 and obtain the peripheral ring 10.

Next, the production method may envisage implementation of all or only some of the steps already described previously with reference to the use of a starting material in the form of a foil, such as electropolishing, anodization, drawing, and possible further treatments designed to modify the surface morphology of the implant.
C) The corneal implant may also be obtained by means of 3D printing of the final implant or else be made of wax from which the implant forming the subject of the present disclosure is then obtained by lost-wax casting. In the latter case, the model made of wax is used, according to the usual techniques of lost-wax casting, for producing the final component of the desired material.
D) The corneal implant may also be made of a material, such as Nitinol, whereby forming is a thermoforming process that becomes thermoforming. In the latter case, the corneal implant may be obtained employing operating conditions for thermoforming known in the art.

### Determination of Young's Modulus of the Corneal Implant

In order to determine the Young's modulus of the device forming the subject of the present invention, it is possible to conduct tests of various types known to the person skilled in the sector, such as tensile tests: 1) on the plate-type specimen made of the same material as that with which the device is then obtained; 2) on a single beam like the ones of which the device is constituted; or else 3) on the final device.
In case 1), it is expedient to use machinery appropriately sized with respect to the ultimate tensile strength of the material being tested; in this case, the load cells that can be used may reach up to 3000 kN.
In case 2), it is expedient to use appropriately sized tensile-testing machinery, equipped with load cells preferably in the range of from 10 N to 10 kN, and it is necessary to have an undulated element representative of the device beam and having a length sufficient for conducting the test, preferably greater than 20 mm.
In case 3) it is possible to use the same modalities as in case 1), albeit taking into account that the load applied in the first part of the tensile test will be used to flatten the dome-shaped device to render it planar and will not be indicative for determining the Young's modulus.

1. Precursor (100) for corneal implant (1) comprising a structural body (2), the structural body (2) comprising:
an outer peripheral ring (10);
an inner peripheral ring (11); and
a connection structure (20) pre-arranged to connect the outer peripheral ring (10) and the inner peripheral ring (11),
wherein said outer peripheral ring (10), said inner peripheral ring (11), and said connection structure (20) lie in one and the same plane, and
wherein said outer peripheral ring (10) and said inner peripheral ring (11) lie at a first distance apart,
said connection structure (20) being designed to undergo a deformation to obtain a predefined dome-like conformation of said structural body (2) wherein said outer peripheral ring (10) lies in a first plane and said inner peripheral ring (11) lies in a second plane distinct from and parallel to said first plane and at a second distance from said outer peripheral ring (10) greater than said first distance,
wherein the connection structure (20) comprises at least one connection element (23) for connecting the outer peripheral ring to the inner peripheral ring that extends along a profile (22) that deviates from a connection path of minimum length, equal to said first distance, so as to have a length in excess of said first distance that is such as to cover said second distance between said outer peripheral ring and said inner peripheral ring in said dome-like conformation of said structural body.

2. Precursor (100) for corneal implant (1) according to claim 1, wherein said profile (22) of said connection element (23) comprises at least one curvilinear stretch.

3. Precursor (100) for corneal implant (1) according to claim 1, wherein the connection structure (20) comprises a plurality of connection elements (23) each comprising a first end (31) connected to the outer peripheral ring (10) and a second end (32) connected to the inner peripheral ring (11).

4. Precursor (100) for corneal implant (1) according to claim 3, wherein said profile (22) is a profile selected from among: sinusoidal (30), arched (40), omega-shaped (50), ring-shaped (60), or combinations thereof.

5. Precursor (100) for corneal implant (1) according to claim 3, wherein said plurality of connection elements (23) have profiles that define one or more corresponding cavities oriented in one and the same direction of rotation with respect to the central axis.

6. Precursor (100) for corneal implant (1) according to claim 1, wherein the connection structure (20) comprises a single connection element (23), which comprises a first end (31) connected to the outer peripheral ring (10) and a second end (32) connected to the inner peripheral ring (11).

7. Precursor (100) for corneal implant (1) according to claim 6, wherein said connection element (23) develops according to a spiral path (70).

8. Precursor (100) for corneal implant (1) according to claim 1, wherein the connection structure (20) further comprises at least one bridging structure (26) for connecting two adjacent elements of said plurality of connection elements (23) or else two distinct and separate points of said connection element (23).

9. Corneal implant (1) configured to correct irregularities of the curvature of the cornea of a subject, the implant (1) having a dome-shaped structural body (2) designed to impose a predefined curvature upon the corneal portions that are to be in contact with the implant, the structural body (2) comprising:
an outer peripheral ring (10) having a central axis, the outer peripheral ring (10) lying in a first plane;
an inner peripheral ring (11) set around the central axis in a second plane offset with respect to the first plane along the central axis; and
a connection structure (20) pre-arranged to connect the outer peripheral ring (10) and the inner peripheral ring (11), the connection structure (20) including at least one connection element (23) that extends along a path connecting the outer peripheral ring (10) to the inner peripheral ring (11),
the connection element (23) having, in a projection plane parallel to the first plane or the second plane, a profile (22) comprising at least one curvilinear stretch.

10. Corneal implant (1) according to claim 9, wherein the connection structure (20) comprises a plurality of connection elements (23), each comprising a first end (31) connected to the outer peripheral ring (10) and a second end (32) connected to the inner peripheral ring (11), wherein each element of said plurality of connection elements (23) extends along a path connecting the outer peripheral ring to the inner peripheral ring and has, in a projection plane parallel to the first plane or the second plane, a profile (22) comprising at least one curvilinear stretch.

11. Corneal implant (1) according to claim 10, wherein said profile (22) of said plurality of connection elements (23) is a profile selected from among: sinusoidal (30), arched (40), omega-shaped (50), ring-shaped (60), or combinations thereof.

12. Corneal implant (1) according to claim 11, wherein said plurality of connection elements (23) has profiles that define one or more corresponding cavities oriented in one and the same direction of rotation with respect to the central axis.

13. Corneal implant (1) according to claim 9, wherein the connection structure (20) comprises a single connection element (23), which comprises a first end (31) connected to the outer peripheral ring (10) and a second end (32) connected to the inner peripheral ring (11),
said connection element (23) extending along a path connecting the outer peripheral ring to the inner peripheral ring and having, in a projection plane parallel to the first plane or the second plane, a profile (22) comprising at least one curvilinear stretch, wherein, preferably, said connection element (23) develops according to a spiral path (70).

14. Corneal implant (1) according to claim 9, wherein the connection structure (20) further comprises at least one bridging structure (26) for connecting two adjacent elements of said plurality of connection elements (23) or else two distinct and separate points of said connection element (23).

15. Corneal implant (1) configured to correct irregularities of the curvature of the cornea of a subject, the implant being obtained from a precursor according to claim 1 through a process for forming the structural body (2), wherein said connection structure undergoes a deformation such that said outer peripheral ring (10) lies in a first plane and said inner peripheral ring (11) lies in a second plane distinct from and parallel to said first plane, and said structural body (2) as a whole assumes a predefined dome-like conformation.
